(19) United States
(12) Patent Application Publication
(10) Pub. No.: US 2026/0029339 A1
(43) Pub. Date: Jan. 29, 2026

(54) PREDICTION DEVICE, PREDICTION SYSTEM, AND PREDICTION PROGRAM

(71) Applicant: KONICA MINOLTA, INC., Chiyoda-ku, Tokyo (JP)

(72) Inventors: Yukako TAKA, Tokyo (JP); Kazuma ODA, Tokyo (JP); Miyuki OKANIWA, Tokyo (JP); Shigeru KOJIMA, Tokyo (JP)

(73) Assignee: KONICA MINOLTA, INC., Chiyoda-ku, Tokyo (JP)

(21) Appl. No.: 18/848,923

(22) PCT Filed: Mar. 8, 2023
(86) PCT No.: PCT/JP2023/008749

§ 371 Date: Mar. 31, 2025

(30) Foreign Application Priority Data
Mar. 24, 2022 (JP) 2022-047925

(51) Int. Cl. G01N 21/3563; G01N 33/44; G16C 60/00

(52) U.S. Cl. CPC G01N 21/3563; G01N 33/442; G16C 60/00

(57) ABSTRACT

A prediction device includes an acquirer that acquires first measurement information and second measurement information obtained by measuring a material property of a composite resin material containing a fibrous material, and a predictor that predicts interfacial shear strength of the composite resin material based on the acquired first measurement information and the acquired second measurement information.

### TECHNICAL FIELD

The present invention relates to a prediction device, a prediction system, and a prediction program.

### BACKGROUND ART

Composite resin materials containing a fibrous material and a resin are excellent in strength and rigidity, and are widely used in electric and electronic applications, civil engineering and construction applications, automobile applications, aircraft applications, and the like. One of important properties of a composite resin material is interfacial shear strength (for example, Patent Literature 1). The interfacial shear strength affects the mechanical properties of the composite resin material.

### CITATION LIST

### Patent Literature

Patent Literature 1: JP 2015-101651 A

### SUMMARY OF INVENTION

### Technical Problem

It is desired to predict the interfacial shear strength of such a composite resin material containing a fibrous material.

The present invention has been made in view of the above-described circumstances, and an object thereof is to provide a prediction device, a prediction system, and a prediction program capable of predicting interfacial shear strength of a composite resin material containing a fibrous material.

### Solution to Problem

The above object of the present invention is achieved by the following means.

(1) A prediction device including: an acquirer that acquires first measurement information and second measurement information obtained by measuring a material property of a composite resin material containing a fibrous material; and a predictor that predicts interfacial shear strength of the composite resin material based on the acquired first measurement information and the acquired second measurement information.

(2) The prediction device according to (1), further including a controller that causes an output unit to output information regarding the predicted interfacial shear strength.

(3) The prediction device according to (1) or (2), in which the first measurement information and the second measurement information include information regarding an interaction between the fibrous material and a resin in the composite resin material.

(4) The prediction device according to any one of (1) to (3), in which the first measurement information is generated by a first measurement device, and the second measurement information is generated by a second measurement device different from the first measurement device.

(5) The prediction device according to (4), in which at least one of the first measurement device and the second measurement device is a measurement device using an electromagnetic wave or an ultrasonic wave.

(6) The prediction device according to (5), in which the first measurement device is an infrared spectrometer.

(7) The prediction device according to (6), in which the second measurement device is an ultrasonic measurement device, a terahertz wave spectrometer, or an impedance spectrometer.

(8) The prediction device according to any one of (1) to (7), in which the acquirer further acquires third measurement information obtained by measuring the material property of the composite resin material, and the predictor predicts the interfacial shear strength based on the acquired first measurement information, the acquired second measurement information, and the acquired third measurement information.

(9) The prediction device according to any one of (1) to (8), in which the predictor predicts the interfacial shear strength by using a trained discriminator.

(10) The prediction device according to (9), further including an extractor that extracts a feature from each of the acquired first measurement information and the acquired second measurement information, in which the predictor predicts the interfacial shear strength using the extracted feature as an input.

(11) The prediction device according to (10), in which the discriminator is subjected to machine learning using the feature as input data and using the interfacial shear strength as output data.

(12) The prediction device according to any one of (1) to (11), in which the material property of the composite resin material is nondestructively measured.

(13) The prediction device according to any one of (1) to (12), further including a calculator that calculates mechanical strength of the composite resin material based on the predicted interfacial shear strength.

(14) A prediction system including: a measurement device that measures a material property of a composite resin material containing a fibrous material; and the prediction device according to any one of (1) to (13).

(15) A prediction program for causing a computer to execute a process including: a step (a) of acquiring first measurement information and second measurement information obtained by measuring a material property of a composite resin material containing a fibrous material; and a step (b) of predicting interfacial shear strength of the composite resin material based on the first measurement information and the second measurement information acquired in the step (a).

### Advantageous Effects of Invention

A prediction device, a prediction system, and a prediction program according to the present invention acquire first measurement information and second measurement information obtained by measuring a material property of a composite resin material containing a fibrous material, and predict interfacial shear strength of the composite resin material based on the acquired first measurement information and the acquired second measurement information. Therefore, it is possible to predict the interfacial shear strength of the composite resin material containing the fibrous material.

### BRIEF DESCRIPTION OF DRAWINGS

FIG. 1 is a diagram illustrating an overall configuration of a prediction system according to an embodiment.

FIG. 2 is a block diagram illustrating a schematic configuration of a prediction device.

FIG. 3 is a block diagram illustrating a functional configuration of the prediction device.

FIG. 4 is a diagram illustrating an example of a display form of information output by the prediction device.

FIG. 5 is a flowchart illustrating a procedure of a prediction process executed in the prediction device.

FIG. 6 is a flowchart illustrating a machine learning method for a trained model.

FIG. 7 is a diagram illustrating a configuration of a prediction system according to Modification Example 1.

FIG. 8 is a block diagram illustrating a functional configuration of a prediction device according to Modification Example 2.

### DESCRIPTION OF EMBODIMENTS

Hereinafter, embodiments of the present invention will be described with reference to the accompanying drawings. In the description of the drawings, the same components are denoted by the same reference signs, and redundant descriptions are omitted. In addition, dimensional ratios in the drawings are exaggerated for convenience of description and may be different from actual ratios.

### EMBODIMENT

### <Configuration of Prediction System>

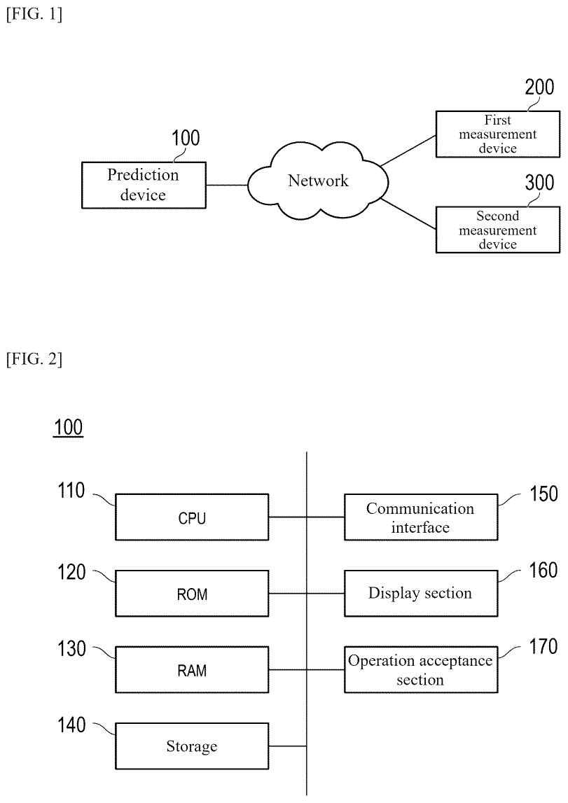

FIG. 1 is a diagram illustrating an overall configuration of a prediction system.

As illustrated in FIG. 1, the prediction system includes, for example, a prediction device 100, a first measurement device 200, and a second measurement device 300. This prediction system predicts interfacial shear strength of a composite resin material containing a fibrous material and a resin. Specifically, the interfacial shear strength between the fibrous material and the resin is predicted.

The resin contained in the composite resin material is, for example, a known thermosetting resin, thermoplastic resin, or the like. Specific examples of the resin contained in the composite resin material include polypropylene resin (PP), polyolefin resins such as maleic anhydride-modified polypropylene (MAHPP), epoxy resin, phenol resin, unsaturated polyester resin, vinyl ester resin, polycarbonate resin, polyester resin, polyamide (PA) resin, liquid crystal polymer resin, polyethersulfone resin, polyether ether ketone resin, polyarylate resin, polyphenylene ether resin, polyphenylene sulfide (PPS) resin, polyacetal resin, polysulfone resin, polyimide resin, polyetherimide resin, polystyrene resin, modified polystyrene resin, AS resin (copolymer of acrylonitrile and styrene), ABS resin (copolymer of acrylonitrile, butadiene, and styrene), modified ABS resin, MBS resin (copolymer of methyl methacrylate, butadiene, and styrene), modified MBS resin, polymethyl methacrylate (PMMA) resin, modified polymethyl methacrylate resin, and the like. The resin contained in the composite resin material may be one of these resins, or may be a mixture of two or more of these resins.

The fibrous material contained in the composite resin material is added to the resin, for example, for the purpose of improving the strength of the composite resin material. Examples of the fibrous material include glass fiber (GF), carbon fiber (CF), aramid fiber, alumina fiber, silicon carbide fiber, boron fiber, silicon carbide fiber, and the like. For the CF, for example, polyacrylonitrile (PAN-based), pitch-based, cellulose-based, or hydrocarbon vapor-grown carbon fibers and graphite fibers can be used. In addition, for the GF, for example, E glass, S glass, and the like can be used. The composite resin material preferably contains at least one of glass fiber (GF) and carbon fiber (CF). Thus, the accuracy of predicting the interfacial shear strength can be improved. The fibrous material contained in the composite resin material may be one of these, or may be a mixture of two or more of these.

### (Prediction Device 100)

The prediction device 100 is, for example, a computer such as a PC, a smartphone, or a tablet terminal. The prediction device 100 functions as a prediction device in the present embodiment. The prediction device 100 is configured to be connectable to the first measurement device 200 and the second measurement device 300, and transmits and receives various kinds of information to and from each of the devices.

FIG. 2 is a block diagram illustrating a schematic configuration of an information processing apparatus.

As illustrated in FIG. 2, the prediction device 100 includes a central processing unit (CPU) 110, a read only memory (ROM) 120, a random access memory (RAM) 130, a storage 140, a communication interface 150, a display section 160, and an operation acceptance section 170. The components are communicably connected to each other via a bus.

The CPU 110 controls the above-described components and performs various kinds of arithmetic processing in accordance with programs recorded in the ROM 120 or the storage 140.

The ROM 120 stores therein various kinds of programs and various kinds of data in advance.

The RAM 130, as a workspace, temporarily stores therein a program and data.

The storage 140 stores various programs including an operating system and various kinds of data. For example, an application for predicting the interfacial shear strength of the composite resin material from first measurement information and second measurement information to be described later using a trained discriminator is installed in the storage 140. Further, the storage 140 may store the first measurement information and the second measurement information acquired from the first measurement device 200 and the second measurement device 300. Further, the storage 140 may store a trained model used as the discriminator and teacher data used for machine learning.

The communication interface 150 is an interface for communicating with the other devices. As the communication interface 150, a communication interface based on various wired or wireless standards is used. The communication interface 150 is used, for example, to receive the first measurement information and the second measurement information from the first measurement device 200 or the second measurement device 300, and to transmit the result of predicting the interfacial shear strength to another apparatus such as a server for storage.

The display section 160 includes a liquid crystal display (LCD), an organic EL display, or the like, and displays various kinds of information. The display section 160 may be configured by viewer software, a printer, or the like. In the present embodiment, the display section 160 functions as an output unit.

The operation acceptance section 170 includes a touch sensor, a pointing device such as a mouse, a keyboard, or the like, and accepts various user operations. Note that the display section 160 and the operation acceptance section 170 may form a touch screen by superimposing the touch sensor as the operation acceptance section 170 on a display surface as the display section 160.

### (First Measurement Device 200 and Second Measurement Device 300)

The first measurement device 200 and the second measurement device 300 are devices for generating the first measurement information and the second measurement information by measuring material properties of the composite resin material, respectively. Here, the material properties include acoustic properties, atomic properties, chemical properties, electrical properties, magnetic properties, mechanical properties, optical properties, radiation properties, thermal properties, and the like. For example, at least one of the first measurement information and the second measurement information includes information regarding the interaction between the fibrous material and the resin in the composite resin material. It is preferable that both the first measurement information and the second measurement information include information regarding the interaction between the fibrous material and the resin in the composite resin material. This is because this interaction is highly likely to have an effect on the interfacial shear strength. It is preferable that the first measurement device 200 and the second measurement device 300 be able to nondestructively measure the material properties of the composite resin material. Thus, the composite resin material after the measurement can be used for a product.

At least one of the first measurement device 200 and the second measurement device 300 is preferably a measurement device that uses an electromagnetic wave or an ultrasonic wave. For example, the first measurement device 200 and the second measurement device 300 are an infrared spectrometer, an impedance spectrometer, a terahertz wave spectrometer, an ultrasonic measurement device, an X-ray diffraction device (XRD), and the like. The first measurement device 200 and the second measurement device 300 are, for example, devices different from each other. It is preferable that the first measurement device 200 be an infrared spectrometer, specifically, a Fourier transform infrared spectroscopy (FTIR), and the second measurement device 300 be an ultrasonic measurement device, a terahertz wave spectrometer, or an impedance spectrometer. As a result, the material properties of the composite resin material can be measured from various perspectives, and the interfacial shear strength of the composite resin material can be predicted with high accuracy.

The first measurement device 200 and the second measurement device 300 generate, for example, an image such as a spectrum of each wavelength or an ultrasonic image including information regarding the interaction between the fibrous material and the resin in the composite resin material. That is, the first measurement information and the second measurement information generated by the first measurement device 200 and the second measurement device 300 are information regarding a spectrum of each wavelength or an image of each wavelength.

The prediction system may further include a third measurement device. The third measurement device is a device for measuring the material properties of the composite resin material to generate third measurement information. The third measurement device is different from the first measurement device 200 and the second measurement device 300. The third measurement device is, for example, a device similar to the first measurement device 200 and the second measurement device 300 exemplified above. The third measurement information preferably includes information regarding the interaction between the fibrous material and the resin in the composite resin material.

### <Functions of Prediction Device 100>

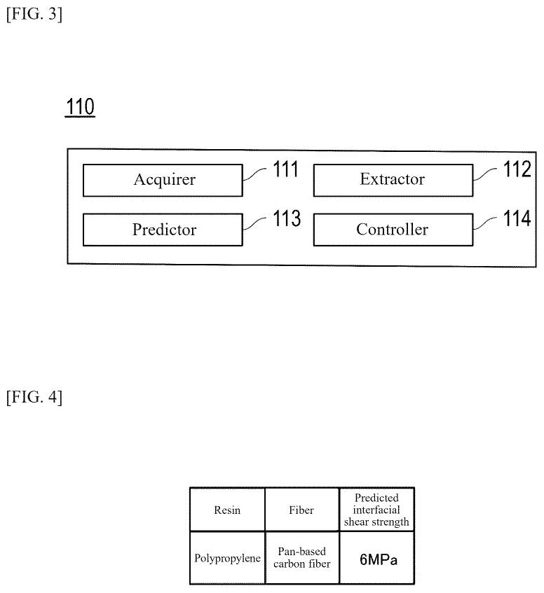

FIG. 3 is a block diagram illustrating a functional configuration of the prediction device 100.

As illustrated in FIG. 3, the prediction device 100 functions as an acquirer 111, an extractor 112, a predictor 113, and a controller 114 when the CPU 110 reads a program stored in the storage 140 and executes processing.

The acquirer 111 acquires the first measurement information generated by the first measurement device 200 and the second measurement information generated by the second measurement device 300. The first measurement information and the second measurement information are information obtained by measuring the material properties of the composite resin material by the first measurement device 200 and the second measurement device 300, respectively. It is preferable that the first measurement information and the second measurement information include information regarding the interaction between the fibrous material and the resin in the fibrous material. The first measurement information is preferably, for example, information regarding an infrared absorption spectrum of the composite resin material.

The extractor 112 extracts a feature from each of the first measurement information and the second measurement information acquired by the acquirer 111. For example, in a case where the first measurement information is information regarding the infrared absorption spectrum of the composite resin material, the extractor 112 extracts a main component from the first measurement information.

In a case where the second measurement information is information regarding a terahertz band absorption spectrum of the composite resin material, the extractor 112 extracts a main component from the second measurement information. In a case where the second measurement information is information regarding to an X-ray diffraction spectrum of the composite resin material, the extractor 112 extracts the main component, crystallinity, or the like from the second measurement information. In a case where the second measurement information is information regarding the spectrum of the composite resin material obtained by impedance spectroscopy of the composite resin material, the extractor 112 extracts capacitance, resistance, or the like from the second measurement information. In a case where the second measurement information is information regarding an ultrasonic image of the composite resin material, the extractor 112 extracts the main component, a frequency characteristic, or the like from the second measurement information. The extractor 112 may extract a plurality of features from each of the first measurement information and the second measurement information.

The acquirer 111 may acquire information from which a feature has been extracted. That is, the first measurement information and the second measurement information may be information in which features have been extracted from information regarding a fiber reinforced resin measured by the first measurement device 200 and the second measurement device 300.

The predictor 113 predicts the interfacial shear strength of the composite resin material containing the fibrous material based on the first measurement information and the second measurement information acquired by the acquirer 111. Specifically, the predictor 113 predicts the interfacial shear strength of the composite resin material by using the trained discriminator and using the features of the first measurement information and the second measurement information extracted by the extractor 112 as inputs.

The controller 114 causes the display section 160 to output information regarding the interfacial shear strength of the composite resin material predicted by the predictor 113.

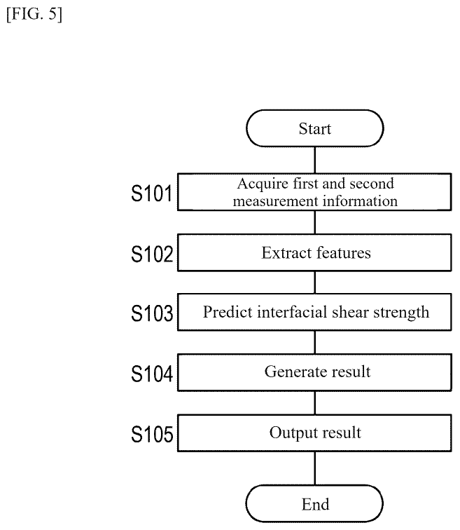

FIG. 4 illustrates an example of the information regarding the interfacial shear strength of the composite resin material output to the display section 160. The display section 160 displays, for example, the value of the predicted interfacial shear strength together with information regarding the composite resin material.

A process executed in the prediction device 100 will be described in detail below.

### <Overview of Process>

FIG. 5 is a flowchart illustrating a procedure of the prediction process executed in the prediction device 100. The process by the prediction device 100 illustrated in the flowchart of FIG. 5 is stored as the program in the storage 140 of the prediction device 100, and is executed by the CPU 110 controlling each section.

### (Step S101)

First, the prediction device 100 acquires the first measurement information obtained by measuring the material properties of the composite resin material by the first measurement device 200 and the second measurement information obtained by measuring the material properties of the composite resin material by the second measurement device 300. For example, the prediction device 100 acquires the first measurement information from the first measurement device 200 and acquires the second measurement information from the second measurement device 300. The first measurement device 200 and the second measurement device 300 may store the first measurement information and the second measurement information to another device such as a server, and the prediction device 100 may acquire the first measurement information and the second measurement information from the other device.

### (Step S102)

The prediction device 100 extracts the features from each of the first measurement information and the second measurement information acquired in the process of step S101.

### (Step S103)

The prediction device 100 inputs the features of each of the first measurement information and the second measurement information extracted in the processing in step S102 to the discriminator that has been subjected to machine learning in advance, and predicts the interfacial shear strength of the composite resin material. For example, the discriminator is subjected to machine learning by a learning method to be described later using teacher data including features of each of first measurement information and second measurement information of a plurality of composite resin materials prepared in advance and a measured value of the interfacial shear strength of each of the plurality of composite resin materials. Specifically, the discriminator is subjected to machine learning with the features of the first measurement information and the second measurement information regarding the plurality of composite resin materials as input data and the measured values of the interfacial shear strength of the plurality of composite resin materials as output data. Accordingly, the prediction device 100 can predict the interfacial shear strength of the composite resin material by inputting the features extracted for each of the first measurement information and the second measurement information to the discriminator. The measured values of the interfacial shear strength of the composite resin materials are acquired using, for example, a microdroplet method, a pinhole method, or a fragmentation method.

The discriminator may be subjected to machine learning using the first measurement information and the second measurement information regarding the plurality of composite resin materials as input data and using the measured values of the interfacial shear strength of each of the plurality of composite resin materials as output data. Furthermore, the information to be input to the discriminator is not limited to the features of each of the first measurement information and the second measurement information. For example, in addition to the features of each of the first measurement information and the second measurement information, information at the time of manufacture may be input to the discriminator and used as information for performing learning and prediction.

### (Step S104)

The prediction device 100 generates a prediction result of the interfacial shear strength of the composite resin material based on the output from the discriminator in the processing in step S103.

### (Step S105)

The prediction device 100 outputs the prediction result generated in the processing in step S104. For example, the prediction device 100 displays the value of the interfacial shear strength of the composite resin material predicted in the processing in step S103 on the display section 160 together with the information regarding the composite resin material (FIG. 4).

### <Learning Processing>

Next, a machine learning method for the trained model used in the discriminator will be described.

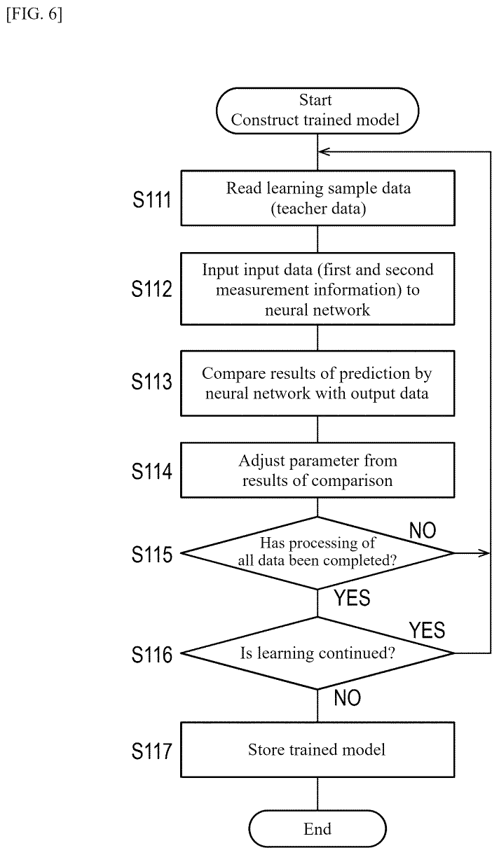

FIG. 6 is a flowchart illustrating the machine learning method for the trained model.

In a process illustrated in FIG. 6, machine learning is executed by using, as learning sample data, a large number (i sets) of data sets in which the features of each of the first measurement information and the second measurement information of the plurality of composite resin materials prepared in advance are used as inputs and the measured values of the interfacial shear strength of each of the plurality of composite resin materials are used as outputs. For example, a stand-alone high-performance computer using processors such as a CPU and a GPU or a cloud computer is used as a learning device (not illustrated) that functions as the discriminator. Hereinafter, a learning method using a neural network formed by combining perceptrons, such as deep learning, in a learning device will be described, but the present invention is not limited thereto, and various methods can be applied. For example, a random forest, a decision tree, a support vector machine (SVMs), logistic regression, a k-nearest neighbor, a topic model, and the like may be applied to the learning method.

### (Step S111)

The learning device reads the learning sample data that is teacher data. In a case where it is the first time, the first set of learning sample data is read. In a case where it is the i-th time, the i-th set of learning sample data is read.

### (Step S112)

The learning device inputs input data among the read learning sample data to the neural network.

### (Step S113)

The learning device compares results of prediction by the neural network with correct data.

### (Step S114)

The learning device adjusts a parameter based on results of the comparison. The learning device adjusts the parameter so as to reduce a difference between the results of the comparison, for example, by executing processing based on back-propagation.

### (Step S115)

The learning device advances the processing to step S116 in a case where the processing of all of the first to i-th sets has been completed (YES), and returns the processing to step S111 in a case where the processing of all of the first to i-th sets has not been completed (NO), reads the next learning sample data, and repeats the processing from step S111.

### (Step S116)

The learning device determines whether to continue learning. In a case where the learning device continues learning (YES), the learning device returns the processing to step S111 and executes the processing on the first to i-th sets again in steps S111 to S115. In a case where the learning device does not continue learning (NO), the learning device advances the processing to step S117.

### (Step S117)

The learning device stores the trained model constructed by the processing so far, and ends the process (end). The storage destination includes an internal memory of the prediction device 100. In the process illustrated in FIG. 5 described above, the interfacial shear strength of the composite resin material is predicted by using the trained model generated in this way.

### <Operational Effects of Prediction Device 100 and Prediction System>

The prediction device 100 and the prediction system according to the present embodiment acquire first measurement information and second measurement information obtained by measuring a material property of a composite resin material containing a fibrous material. Then, the prediction device 100 and the prediction system predict the interfacial shear strength of the composite resin material based on the acquired first measurement information and the acquired second measurement information. Therefore, it is possible to predict the interfacial shear strength of the composite resin material containing the fibrous material. Hereinafter, the operational effects will be described.

As described above, the interfacial shear strength can affect the mechanical properties of the composite resin material. Therefore, it is desirable to know the interfacial shear strength of the composite resin material. The interfacial shear strength of the composite resin material is mainly measured using a microdroplet method. However, in the microdroplet method, one fiber is taken out from a fiber bundle, and the interfacial shear strength is measured. In addition, a method (e.g., JP 2018-146349 A) has also been proposed in which a plate-shaped sample is cut out from a molded article and then polished and the interfacial shear strength of a composite resin material is evaluated using a nanoindentation method, but this method requires a polishing step, measurements at a plurality of positions, and the like. As described above, it takes time and effort to measure the interfacial shear strength of the composite resin material, and it is difficult to efficiently measure the interfacial shear strength of a large number of composite resin materials.

In contrast, in the prediction system and the prediction device 100 according to the present embodiment, the interfacial shear strength of the composite resin material is predicted based on the first measurement information and the second measurement information obtained by measuring the material properties of the composite resin material containing the fibrous material. Therefore, direct measurement of the interfacial shear strength by a microdroplet method or the like is not required. Therefore, for example, the user can cut out the molded article and measure the material properties of the composite resin material with the first measurement device 200 and the second measurement device 300, and can easily ascertain the interfacial shear strength.

Furthermore, the interfacial shear strength of the composite resin material is predicted based on a plurality of pieces of measurement information, thus allowing more versatile prediction of the interfacial shear strength than prediction based on a single piece of measurement information. Therefore, it becomes possible to predict the interfacial shear strength with higher accuracy.

Furthermore, by using an infrared spectrometer as the first measurement device 200, it is possible to effectively acquire information regarding the interaction between the fibrous material and the resin in the composite resin material. This makes it possible to predict the interfacial shear strength with higher accuracy. In addition, it is possible to improve the accuracy of predicting the interfacial shear strength by using an ultrasonic measurement device, a terahertz wave spectrometer, or an impedance spectrometer as the second measurement device 300.

As described above, the prediction device 100 and the prediction system according to the present embodiment can predict the interfacial shear strength of a composite resin material containing a fibrous material.

Hereinafter, modification examples of the above-described embodiment will be described. A description of a similar configuration to that described in the above embodiment is not repeated.

### Modification Example 1

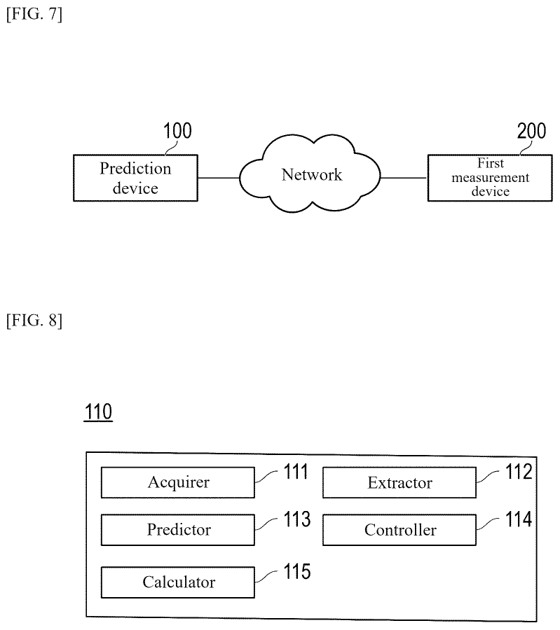

FIG. 7 illustrates a configuration of a prediction system according to Modification Example 1. The prediction system may include the prediction device 100 and one measurement device (the first measurement device 200). That is, the prediction system may not include the second measurement device (the second measurement device 300 illustrated in FIG. 1). In this prediction system, first measurement information and second measurement information are generated by the first measurement device 200.

The prediction device 100 extracts a feature from each of the first measurement information and the second measurement information. In other words, in a case where the prediction device 100 can extract a plurality of features, the first measurement information and the second measurement information may be generated by a single measurement device (the first measurement device 200). For example, in a case where the first measurement device 200 is an X-ray diffraction device, the prediction device 100 extracts, as a feature, the main component and the crystallinity of the X-ray diffraction spectrum.

### Modification Example 2

FIG. 8 illustrates a functional configuration of a prediction device 100 in a prediction system according to Modification Example 2. The prediction device 100 may function as a calculator 115 in addition to the acquirer 111, the extractor 112, the predictor 113, and the controller 114.

The calculator 115 calculates the mechanical strength of the composite resin material based on the interfacial shear strength of the composite resin material predicted by the predictor 113. The calculator 115 calculates the mechanical strength of the composite resin material using, for example, machine learning. The calculator 115 preferably calculates the mechanical strength of the composite resin material based on the interfacial shear strength of the composite resin material predicted by the predictor 113 and information regarding the fiber amount, the fiber diameter, and the like of the composite resin material. Thus, the accuracy of predicting the mechanical strength can be improved. For example, the controller 114 causes the display section 160 to output information regarding the mechanical strength calculated by the calculator 115 together with information regarding the interfacial shear strength predicted by the predictor 113.

### EXAMPLES

The effects of the present invention will be described using the following Examples. However, the technical scope of the present invention is not limited to the following Examples alone.

### (Creation of Trained Discriminator)

First, samples of 12 types of composite resin materials for creating teacher data were prepared. The samples were prepared by combining four kinds of resins and three kinds of fibers indicated below. The fiber concentration was set to 20%. The resins and the fibers were mixed in advance at desired ratios using a Labo Plastomill (registered trademark) extruder manufactured by Toyo Seiki Seisaku-sho, Ltd. Thus, pellets were produced. The samples of the 12 types of composite resin materials were molded using an injection molding machine SE50D manufactured by Sumitomo Heavy Industries, Ltd. The shape of each of the samples was a dumbbell-shaped test piece type A1 shown in JIS K7139.

The resins: polypropylene (Noblen W101, manufactured by Sumitomo Chemical Co., Ltd.), polyamide 6 (Amilan CM1017, manufactured by Toray Industries, Inc.), ABS (Toyolac 700 to 314, manufactured by Toray Industries, Inc.), and polycarbonate (Iupilon H-3000R, manufactured by Mitsubishi Engineering-Plastics Corporation):

The fibers: PAN (polyacrylonitrile)-based carbon fibers (Torayca T700SC, manufactured by Toray Industries, Inc.), PAN-based carbon fibers (IM600, manufactured by Teijin Limited), and PAN-based carbon fibers (Torayca T1000 GB, manufactured by Toray Industries, Inc.).

Next, each of the samples of the 12 types of composite resin materials was measured using the following measurement devices, and features thereof were learned by the discriminator. The measurement was performed near the center of the dumbbell-shaped test piece.

An X-ray diffraction device (Smart Lab manufactured by Rigaku Corporation): an FT-IR (Fourier transform infrared spectroscopy) device (AVATAR370 manufactured by Thermo Fisher Scientific Co., Ltd.); a terahertz-wave spectrometer (C12068-01 manufactured by Hamamatsu Photonics K. K); an ultrasonic imaging device; and an impedance spectrometer (Model 126096 manufactured by Solartron). Conductive tapes having diameters of 5 mm were attached to two portions of each of the samples with a gap of 50 mm therebetween, and the samples were measured using the tapes as electrodes).

Furthermore, the interfacial shear strength of each of the samples of the 12 types of composite resin materials was measured using a microdroplet (MD) method or a pinhole method, and the discriminator was caused to learn the interfacial shear strength. In the microdroplet method, MODEL HM410 manufactured by Tohei Sangyo Co., Ltd. was used, and the interfacial shear strength was measured at the dissolution temperature of each of the resins. In the pinhole method, a pinhole-type tester manufactured by Shinsousha Co., Ltd. was used, and the interfacial shear strength was measured at a desired temperature. The pinhole method was used to measure the interfacial shear strength of each of the composite resin materials at different temperatures (e.g., the interfacial shear strength of a composite resin material containing polyamide 6 at 245° C. and 260° C.). In the microdroplet method, a composite resin material containing polypropylene was measured at 215° C., a composite resin material containing polyamide 6 was measured at 220° C., a composite resin material containing ABS was measured at 230° C., and a composite resin material containing polycarbonate was measured at 285° C.

### Examples 1 to 9 and Comparative Example

First, samples of 2 types of composite resin materials were produced. The samples were prepared by combining two kinds of resins and one kind of fiber as shown below. The samples were formed in a manner similar to that of the above-described teacher data.

The resins: polypropylene (Noblen W101 manufactured by Sumitomo Chemical Co., Ltd.), polyamide 6 (Amilan CM1017 manufactured by Toray Industries, Inc.):

The fiber: PAN-based carbon fibers (Torayca T700SC manufactured by Toray Industries, Inc.).

The samples of the 2 types of composite resin materials were measured using measurement devices indicated in Table 1 below. Thereafter, features of spectra or images measured by the measurement devices were input to the trained discriminator to obtain a predicted value of the interfacial shear strength.

In addition, the interfacial shear strength of each of the above-described 4 types of composite resin materials was measured using the microdroplet method or the pinhole method, and the measured values were obtained. Next, errors between the predicted value and the measured values were calculated using the following Equation (1), and then an average of the errors of the 2 types of composite resin materials was obtained. In the following Table 1, the average of the errors of the 2 types of composite resin materials in Comparative Examples was set to 1, and the averages in Examples 1 to 9 were described as relative values. That is, the smaller the value in a column of “error” in Table 1, the higher the accuracy of the interfacial shear strength predicted using the trained discriminator.

$$\begin{matrix}[ Mathematical Formula 1 ] & \\ Error = \vert ( predicted value - measured value ) / measured value \vert & ( 1 )\end{matrix}$$

TABLE 1

| | Method for | | | | | | | |
|---|---|---|---|---|---|---|---|---|
| | obtaining | First | | Second | | Third | | |
| | measured | Measurement | | Measurement | | Measurement | | |
| | value | device | Feature | device | Feature | device | Feature | Error |
| Example 1 | MD | FT-IR | Main | Terahertz | Main | | | 0.4 |
| | method | | component | | component | | | |
| Example 2 | MD | FT-IR | Main | Ultrasonic | Main | | | 0.4 |
| | method | | component | wave | component | | | |
| Example 3 | MD | FT-IR | Main | Impedance | Capacity | | | 0.5 |
| | method | | component | spectroscopy | | | | |
| Example 4 | MD | FT-IR | Main | XRD | Main | | | 0.6 |
| | method | | component | | component | | | |
| Example 5 | MD | Terahertz | Main | Ultrasonic | Main | | | 0.7 |
| | method | | component | wave | component | | | |
| Example 6 | Pinhole | FT-IR | Main | Terahertz | Main | | | 0.4 |
| | | | component | | component | | | |
| Example 7 | MD | Ultrasonic | Main | Ultrasonic | Frequency | | | 0.7 |
| | method | wave | component | wave | characteristic | | | |
| Example 8 | MD | XRD | Main | XRD | Crystallinity | | | 0.8 |
| | method | | component | | | | | |
| Example 9 | MD | FT-IR | Main | Terahertz | Main | Ultrasonic | Main | 0.3 |
| | method | | component | | component | wave | component | |
| Comparative | MD | FT-IR | Main | | | | | 1 |
| Example | method | | component | | | | | |

The errors were smaller in Examples 1 to 9 in which a plurality of features were input to the discriminator than that in Comparative Example. Furthermore, among Examples 1 to 9, in Examples 1 to 4, 6, and 9 in which FT-IR was used, the errors could be reduced as compared with Examples 5 and 8. Furthermore, the interfacial shear strength could be predicted with higher accuracy by inputting three types of features to the discriminator (Example 9).

The configurations of the prediction device 100 and the prediction system described above are merely main configurations for describing the features of the above-described embodiment and Examples, and can be variously modified without being limited to the above-described configurations within the scope of the claims. In addition, a configuration included in a general prediction system is not excluded.

For example, the prediction device 100 may include constituent elements other than the above-described constituent elements, or may not include some of the above-described constituent elements.

Further, each of the prediction device 100, the first measurement device 200, and the second measurement device 300 may be constituted by a plurality of devices or may be constituted by a single device.

Further, the function of each component may be implemented by another component. For example, the first measurement device 200 or the second measurement device 300 may be integrated into the prediction device 100, and some or all of the functions of the first measurement device 200 and the second measurement device 300 may be implemented by the prediction device 100.

Also, the processing units of the flowcharts in the above embodiment are divided according to the main processing contents in order to facilitate understanding of each processing. The present invention is not limited by how the processing steps are classified. Each processing can be further divided into more processing steps. In addition, one processing step may execute more processes.

Means and methods for performing various kinds of processing in the system according to the above-described embodiment can be implemented by any of a dedicated hardware circuit and a programmed computer. The program may be provided, for example, by a computer-readable recording medium such as a flexible disk and a CD-ROM, or may be provided online via a network such as the Internet. In this case, the program recorded on the computer-readable recording medium is usually transferred to and stored in a storage section such as a hard disk. Furthermore, the program may be provided as independent application software, or may be incorporated in software of the device as one function of the system.

The present application is based on Japanese Patent Application No. 2022-47925 filed on Mar. 24, 2022, the disclosure content of which is incorporated by reference in its entirety.

### REFERENCE SIGNS LIST

| | |
|---|---|
| 100 | prediction device |
| 110 | CPU |
| 111 | acquirer |
| 112 | extractor |
| 113 | predictor |
| 114 | controller |
| 115 | calculator |
| 120 | ROM |
| 130 | RAM |
| 140 | storage |
| 150 | communication interface |
| 160 | display section |
| 170 | operation acceptance section |
| 200 | first measurement device |
| 300 | second measurement device |

1. A prediction device comprising:
a hardware processor that:
acquires first measurement information and second measurement information obtained by measuring a material property of a composite resin material containing a fibrous material; and
predicts interfacial shear strength of the composite resin material based on the acquired first measurement information and the acquired second measurement information.

2. The prediction device according to claim 1, wherein the hardware processor causes an outputter to output information regarding the predicted interfacial shear strength.

3. The prediction device according to claim 1, wherein the first measurement information and the second measurement information include information regarding an interaction between the fibrous material and a resin in the composite resin material.

4. The prediction device according to claim 1, wherein the first measurement information is generated by a first measurement device, and the second measurement information is generated by a second measurement device different from the first measurement device.

5. The prediction device according to claim 4, wherein at least one of the first measurement device and the second measurement device is a measurement device using an electromagnetic wave or an ultrasonic wave.

6. The prediction device according to claim 5, wherein the first measurement device is an infrared spectrometer.

7. The prediction device according to claim 6, wherein the second measurement device is an ultrasonic measurement device, a terahertz wave spectrometer, or an impedance spectrometer.

8. The prediction device according to claim 1, wherein the hardware processor further acquires third measurement information obtained by measuring the material property of the composite resin material, and the hardware processor predicts the interfacial shear strength based on the acquired first measurement information, the acquired second measurement information, and the acquired third measurement information.

9. The prediction device according to claim 1, wherein the hardware processor predicts the interfacial shear strength using a trained discriminator.

10. The prediction device according to claim 9, wherein the hardware processor extracts a feature from each of the acquired first measurement information and the acquired second measurement information, and predicts the interfacial shear strength using the extracted feature as an input.

11. The prediction device according to claim 10, wherein the discriminator is subjected to machine learning using the feature as input data and the interfacial shear strength as output data.

12. The prediction device according to claim 1, wherein the material property of the composite resin material is nondestructively measured.

13. The prediction device according to claim 1, wherein the hardware processor calculates mechanical strength of the composite resin material based on the predicted interfacial shear strength.

14. A prediction system comprising:
a measurement device that measures a material property of a composite resin material containing a fibrous material; and
the prediction device according to claim 1.

15. A non-transitory recording medium storing a computer readable prediction program for causing a computer to perform operations comprising:
acquiring first measurement information and second measurement information obtained by measuring a material property of a composite resin material containing a fibrous material; and
predicting interfacial shear strength of the composite resin material based on the acquired first measurement information and the acquired second measurement information.
